(19) United States
(12) Patent Application Publication
(10) Pub. No.: US 2021/0205560 A1
(43) Pub. Date: Jul. 8, 2021

(54) POSITIVE AIRWAY PRESSURE (PAP) DEVICE WITH BUY NOW BUTTON

(71) Applicant: KONINKLIJKE PHILIPS N.V., Eindhoven (NL)

(72) Inventors: Richard Thomas HAIBACH, Verona, PA (US); Daniel STEED, North Huntingdon, PA (US)

(21) Appl. No.: 17/128,513

(22) Filed: Dec. 21, 2020

Related U.S. Application Data
(60) Provisional application No. 62/953,246, filed on Dec. 24, 2019.

(51) Int. Cl. A61M 16/00; A61M 16/06; A61M 16/08; G06F 3/0488

(52) U.S. Cl. CPC A61M 16/024; A61M 16/0683; A61M 2205/505; G06F 3/0488; A61M 16/0875

(57) ABSTRACT

A pressure generating device structured to be operatively coupled to a system for delivering a flow of pressurized breathing gas to a patient includes a controller and a touch screen with a graphical user interface (GUI). The system comprises a number of accessory components, and the controller is structured to monitor a number of factors related to determining when the pressure generating device and each of the number of accessory components should be replaced. A method for facilitating replacement of individual components for the system utilizes the pressure generating device.

### CROSS-REFERENCE TO PRIOR APPLICATIONS

This application claims the benefit of U.S. Provisional Application No. 62/953,246, filed on 24 Dec. 2019. This application is hereby incorporated by reference herein.

### BACKGROUND OF THE INVENTION

### 1. Field of the Invention

The present invention pertains to pressurized breathing gas systems, and more particularly, to simplifying the process of replacing components used with pressurized breathing gas systems based on conditions such as age and usage of the components.

### 2. Description of the Related Art

Many individuals suffer from disordered breathing during sleep. Sleep apnea is a common example of such sleep disordered breathing suffered by millions of people throughout the world. It is known to deliver positive airway pressure (PAP) to treat a medical disorder, such as chronic obstructive pulmonary disease (COPD) or sleep apnea syndrome, in particular, obstructive sleep apnea (OSA). Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle.

Pressurized breathing gas therapies such as CPAP involve the placement of a patient interface device including a mask component on the face of a patient. The mask component may be, without limitation, a nasal mask that covers the patient's nose, a nasal cushion having nasal prongs that are received within the patient's nares, a nasal/oral mask that covers the nose and mouth, or a full face mask that covers the patient's face. The patient interface device interfaces the ventilator or pressure support device with the airway of the patient, so that a flow of breathing gas can be delivered from a pressure/flow generating device to the airway of the patient. It is known to maintain such devices on the face of a wearer by a headgear having one or more straps adapted to fit over/around the patient's head.

Ideally, users of pressurized breathing gas therapy systems use their systems every time they sleep, i.e. daily. The frequent use of these systems necessitates that each of the components, such as the nasal and/or oral mask, the nasal cushion, and the frame, among others, needs to be replaced periodically to maintain the hygiene and optimal performance of the system. Currently, the user must track the age and usage level of each component in the system. A user may not be aware of the need to replace a component regularly, or of the availability of a new and improved component. Accordingly, there is room for improvement in systems and methods for replacing the components of pressurized breathing gas therapy systems.

### SUMMARY OF THE INVENTION

Accordingly, one or more embodiments provide an apparatus structured to: track the age and frequency of use of various components included in a pressurized breathing gas therapy system, inform a user when each component is due to be replaced, and provide a mechanism for purchasing replacement components using a pressure generating device operatively coupled to the system. In one embodiment, a pressure generating device structured to be operatively coupled to a system for delivering a flow of pressurized breathing gas to a patient is provided. The pressure generating device includes a controller and a touch screen with a graphical user interface (GUI). The system comprises a number of accessory components, and the controller is structured to monitor a number of factors related to determining when the pressure generating device and each of the number of accessory components should be replaced.

A method for facilitating a purchase of replacement components for a system used for delivering a flow of pressurized breathing gas to a patient includes: providing a pressure generating device operatively coupled to the system, wherein the pressure generating device includes a controller and a touch screen with a GUI; and monitoring, with the controller, a number of factors related to determining when a one or more individual components used with the system should be replaced. The one or more individual components includes the pressure generating device and a number of accessory components.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 is a schematic diagram of a pressurized breathing gas system according to an exemplary embodiment of the disclosed concept; and

FIG. 2 is an illustration of a message displayed on a GUI of a pressure generating device according to an exemplary embodiment of the disclosed concept.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

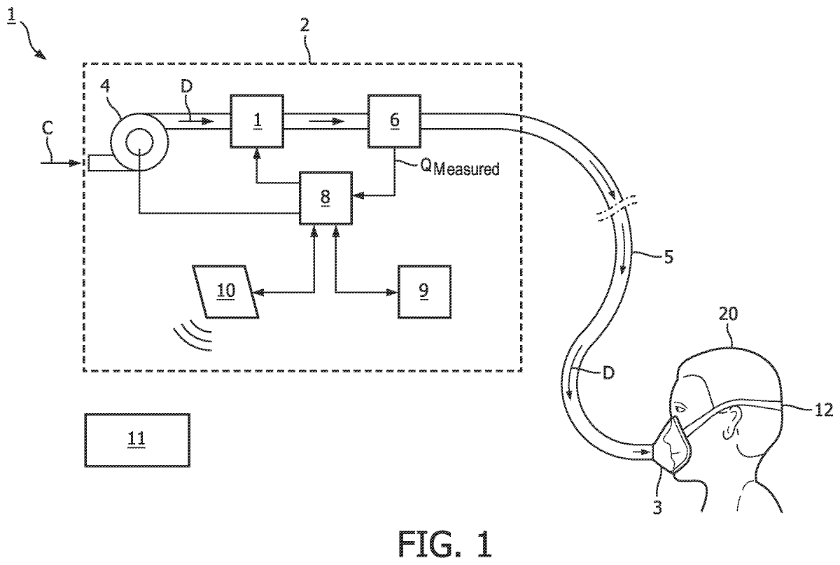

FIG. 1 shows a schematic diagram of a pressurized breathing gas system 1. Pressurized breathing gas system 1 includes a pressure generating device 2 for delivering a flow of breathing gas to a patient 20 through a mask 3, which is typically worn by or otherwise attached to patient 20 to communicate the flow of breathing gas to the airway of patient 20. In the illustrated exemplary embodiment show in FIG. 1, mask 3 is a nasal/oral mask. It will be appreciated, however, that mask 3 can be a nasal mask, a pillows style nasal cushion, a cradle style nasal cushion, a full face mask, or any other patient interface device that provides a suitable gas flow communicating function without departing from the scope of the present invention. Mask 3 may be secured to the head of patient 20 by a strap 12. It will be appreciated, however, that strap 12 may be excluded from pressurized breathing gas system 1 and mask 3 may be secured to the head of patient 20 by other means without departing from the scope of the disclosed concept.

Pressure generating device 2 includes a gas flow generator 4, such as a blower used in a conventional CPAP or bi-level pressure support device, which receives breathing gas, generally indicated by arrow C, from any suitable source, e.g., a pressurized tank of oxygen or air, the ambient atmosphere, or a combination thereof. Gas flow generator 4 generates a flow of breathing gas, such as air, oxygen, or a mixture thereof, for delivery to an airway of patient 20 at relatively higher and lower pressures, i.e., generally equal to or above ambient atmospheric pressure. The pressurized flow of breathing gas, generally indicated by arrow D from gas flow generator 4, is delivered via a delivery conduit 5 to mask 3.

Pressurized breathing gas system 1 further includes flow sensor 6 that measures the flow of the breathing gas within delivery conduit 5. In the particular embodiment shown in FIG. 1, flow sensor 6 is interposed in line with delivery conduit 5, most preferably downstream of valve 7, which controls pressure. Flow sensor 6 generates a flow signal Q_{MEASURED} that is provided to controller 8 and is used by controller 8 to determine the rate of flow of gas at patient 20, referred to as Q_{PATIENT}. It will be appreciated that a pressure generating device 2 may employ other configurations of pressure control and flow sensing without departing from the scope of the disclosed concept.

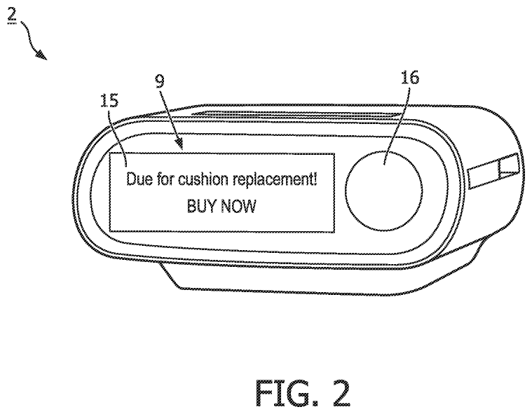

Controller 8 includes a processing unit, such as, for example, a microprocessor, a microcontroller or some other suitable processing device, and a memory (that is provided as part of the processing unit or that is operatively coupled to the processing unit) that provides a tangible storage medium for data and software routines executable by the processing unit for controlling the operation of pressurized breathing gas system 1. Input/output unit 9 is provided for setting various parameters used by pressurized breathing gas system 1, as well as for displaying and outputting information and data to a user, such as a clinician or caregiver. Input/output unit 9 includes a graphical user interface (GUI) 15, shown in FIG. 2. In an exemplary embodiment, input/output unit 9 also includes a touch screen. It will be appreciated, however, that input/output unit 9 could include physical buttons or turn knobs 16 instead of or in addition to a touch screen, or any other means for enabling a user to enter input into input/output unit 9 without departing from the scope of the disclosed concept.

Pressure generating device 2 further includes a communication module 10.

Communication module 10 enables pressure generating device 2 to connect to the internet either directly, or indirectly, by communicating with an internet module 11. Communication module 10 may comprise short range wireless technology, broadband technology, or any other data communication technology which permits communication between two devices or enables a device to connect to the internet without departing from the scope of the disclosed concept. In an exemplary embodiment where communication module 10 connects to the internet indirectly via communicating with internet module 11, internet module 11 may be a personal computer. However, it will be appreciated that internet module 11 may be any other device capable of connecting to the internet without departing from the scope of the disclosed concept. It will also be appreciated that, if communication module 10 is capable of connecting directly to the internet, internet module 11 may be excluded from pressurized breathing gas system 1 without departing from the scope of the disclosed concept.

It is an objective of the disclosed concept to simplify the process of replacing components for a pressurized breathing gas system 1, and this objective is facilitated by the use of input/output unit 9, communication module 10, and internet module 11. Components of pressurized breathing gas system 1 that may need to be replaced include, for example and without limitation: mask 3, delivery conduit 5, strap 12, and pressure generating device 2. It is contemplated that every time a component of pressurized breathing gas system 1 is replaced, patient 20 or another person can input information about the replacement component to controller 8 via input/output unit 9. For example and without limitation, such information about the replacement may include the date of replacement and the make/model of the replacement component. Controller 8 subsequently tracks the age and number of uses of each component in pressurized breathing gas system 1, for example and without limitation, by keeping track of how many times pressurized breathing gas system 1 is used after a component has been replaced. The components of pressurized breathing gas system 1 may also include electronic chips such as near field communication (NFC) chips with information about the component encoded within them such that the NFC chips may communicate the encoded information to controller 8, thereby eliminating the need for a user to manually input information about the replacement to controller 8 via input/output unit 9. It will be appreciated, however, that NFC chips may be excluded from the components of pressurized breathing gas system 1 without departing from the scope of the disclosed concept.

FIG. 2 illustrates an example of a “Buy Now” message displayed to a user on GUI 15 indicating that a component is becoming due for replacement based on predetermined parameters regarding hygiene and wear and tear limits of the components. A user may respond to a message displayed on GUI 15 using digital buttons on the touch screen or physical buttons or turn knobs 16 of input/output unit 9. Because controller 8 has internet connectivity, the user would be able to complete a purchase of the replacement component suggested by a “Buy Now” message and authorize payment for the purchase by following a series of prompts generated on GUI 15 by touching the buttons of input/output unit 9 after seeing the “Buy Now” message.

In an exemplary embodiment, information about the health insurance policy of patient 20 could also be entered into input/output unit 9, and controller 8 could display a message on GUI 15 indicating when patient 20 is eligible to have the replacement of a component covered by his or her insurance policy. It is also contemplated that input/output unit 9, communication module 10, and internet module 11 could be used to facilitate the display of advertisements on GUI 15 and that the manufacturer of pressure generating device 2 could solicit advertising revenue from a variety of companies. For example and without limitation, a coffee company may pay the manufacturer of pressure generating device 2 to advertise the coffee company's products on GUI 15 during morning hours, or the manufacturer of pressure generating device 2 may wish to advertise its other products on GUI 15, such as air purifiers, on days when the local air pollution level is high.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although this description includes details for the purpose of illustration based on what is currently considered to be the most practical and preferred embodiments, it is to be understood that such detail is solely for that purpose and that the disclosure is not limited to the disclosed embodiments, but, on the contrary, is intended to cover modifications and equivalent arrangements that are within the spirit and scope of the appended claims. For example, it is to be understood that, to the extent possible, one or more features of any embodiment are contemplated to be combined with one or more features of any other embodiment.

What is claimed is:

1. A pressure generating device structured to be operatively coupled to a system for delivering a flow of pressurized breathing gas to a patient, the pressure generating device comprising:
a controller; and
a touch screen with a graphical user interface (GUI);
wherein the system comprises a number of accessory components, and
wherein the controller is structured to monitor a number of factors related to determining when the pressure generating device and each of the number of accessory components should be replaced.

2. The pressure generating device of claim 1,
wherein the controller is structured to have a connection to the internet.

3. The pressure generating device of claim 1, wherein the controller is structured to make a determination regarding whether the pressure generating device or any of the number of accessory components should be replaced.

4. The pressure generating device of claim 3, wherein the GUI is structured to display a message indicating that the pressure generating device or any of the number of accessory components should be replaced based on the determination.

5. The pressure generating device of claim 1, wherein the number of accessory components comprises:
a patient interface,
a tubing assembly through which pressurized air generated by the pressure generating device may flow, or
an apparatus structured to secure the patient interface to the face of the patient.

6. The pressure generating device of claim 1, wherein the number of factors includes age of the pressure generating device, age of each of the number of accessory components, number of uses of the pressure generating device, and number of uses of each of the number of accessory components.

7. The pressure generating device of claim 1, wherein the number of factors includes eligibility for having an insurance policy of the patient cover a replacement of the pressure generating device or any of the number of accessory components.

8. The pressure generating device of claim 2,
wherein the GUI is structured to display a message prompting a user of the pressure generating device to make a purchase of a replacement pressure generating device or a replacement of any of the number of accessory components,
wherein the GUI is structured to display subsequent prompts in response to the user touching the message on the touch screen, and
wherein the user may complete the purchase by touching the message on the touch screen and following subsequent prompts asking the user for authorization to charge the user for payment.

9. A method for facilitating a purchase of replacement components for a system used for delivering a flow of pressurized breathing gas to a patient, the method comprising:
providing a pressure generating device structured to be operatively coupled to the system, the pressure generating device comprising:
a controller; and
a touch screen with a graphical user interface (GUI); and
monitoring, with the controller, a number of factors related to determining when a one or more individual components used with the system should be replaced,
wherein the one or more individual components includes the pressure generating device and a number of accessory components.

10. The method of claim 9, further comprising:
connecting the controller to the interne.

11. The method of claim 9, further comprising:
making a determination with the controller regarding whether any of the one or more individual components should be replaced, based on the monitoring.

12. The method of claim 11, further comprising:
displaying a message with the GUI indicating that any of the one or more individual components should be replaced based on the determination.

13. The method of claim 9, wherein the number of accessory components includes a patient interface, a tubing assembly through which pressurized air generated by the pressure generating device may flow, or an apparatus structured to secure the patient interface to the face of a patient.

14. The method of claim 9, wherein the number of factors includes age of each of the one or more individual components and number of uses of each of the one or more individual components.

15. The method of claim 9, wherein the number of factors includes eligibility for having an insurance policy of the patient cover a replacement of the pressure generating device or any of the number of accessory components

16. The method of claim 10, further comprising:
displaying a message with the GUI prompting a user of the pressure generating device to make a purchase of the one or more individual components;
displaying subsequent prompts with the GUI in response to the user touching the message; and
completing the purchase after receiving authorization from the user to charge the user for payment in response to the user following the subsequent prompts.
